(19) United States
(12) Patent Application Publication
(10) Pub. No.: US 2026/0021245 A1
(43) Pub. Date: Jan. 22, 2026

(54) TWO PARTS DRIP CHAMBER OF INFUSION SET AND METHOD OF MANUFACTURING TWO PARTS DRIP CHAMBER

(71) Applicant: Nachiket Raghuvir AMIN, SURAT (IN)

(72) Inventor: Nachiket Raghuvir AMIN, SURAT (IN)

(21) Appl. No.: 19/103,580

(22) PCT Filed: May 1, 2024
(86) PCT No.: PCT/IN2024/050460

§ 371 Date: Feb. 13, 2025

(30) Foreign Application Priority Data
Mar. 8, 2024 (IN) 202421016718

(51) Int. Cl. A61M 5/14; A61L 29/04; A61L 29/06

(52) U.S. Cl. CPC A61M 5/1411; A61L 29/042; A61L 29/06; A61M 2207/00

(57) ABSTRACT

The two-part drip chamber includes an upper body made of a material containing one or more of Acrylonitrile Butadiene Styrene (ABS) or Polycarbonate (PC) and a lower body made of a material containing one or more of Thermoplastic Elastomer (TPE), Thermoplastic Polyurethane (TPU) or Poly Vinyl Chloride (PVC). A distal end region of the upper body is preformed in a circular ring shape, and a proximal end region of the lower body is preformed in a circular ring shape. The preformed circular ring shape region at the distal end of the upper body and the preformed circular ring shape region at the proximal end of the lower body are securely coupled in a leak-proof manner by an adhesive material selected from one or more of Cyclohexanone or Cyclohexanone with Poly Vinyl Chloride (PVC) resins, thereby producing an ergonomic elevated grip structure.

### TECHNICAL FIELD OF THE INVENTION

The present invention relates to two parts drip chamber of infusion set. More particularly, the invention relates to a drip chamber with rigid first part or an upper body and flexible second part or a lower body, wherein both parts featuring with end portions preformed in circular ring shape and that are directly connected in a leak-proof manner by adhesive material to administer continuous air free delivery of intravenous fluid to patients. The invention also relates to method for manufacturing two parts drip chamber.

### BACKGROUND OF THE INVENTION

Infusions are performed for therapeutic purposes in human and animal by using intravenous infusion system or set. For example, infusion sets are used to administer controlled amount of intravenous fluids, medications and nutrients directly into a person's body seeking treatment.

Infusion sets typically comprise a flexible infusion tube and a drip chamber. The infusion sets may optionally include additional components, such as a fluid flow controlling device, for example a roller clamp, is provided on the infusion line for regulating the flow rate of the infusion fluid. The fluid to be administered in the course of an infusion or transfusion is provided in a fluid source, such as a container, bag or bottle containing a defined infusion.

The drip chamber is connected to the container via a container connection such that the fluid may pass from the container into the drip chamber. The container connection may, for example, comprise a piercing device, such as a hollow mandrel, which may be used to pierce a septum closing the container. The drip chamber is in fluid communication with one end of the tube such that fluid may enter the tube from the drip chamber. In this sense, the drip chamber is a fluid communication system, as fluid is conducted from the container through the drip chamber into the tube. The tube comprises a standard connector is then connected via conventional access means such as a needle, a catheter or the like to a patient at another end.

The drip chamber usually comprises a venting device with a manually operated or an automatic venting valve. An air channel allows air to enter the container. To prevent contamination when air from the environment flows through the venting valve, the venting device usually comprises a venting filter. A closing mechanism is often provided for the air channel to ensure that liquid cannot pass through to the venting filter and penetrate it, making it impermeable to air. The present invention is compatible with manual and automatic venting devices.

The drip chambers known in the prior art and available in the market usually comprise a short and not highly transparent upper part and a pumpable lower part. Some of the drip chambers available in the market also have a grip ring formed in the area of the outer wall of the drip chamber. The grip ring forms an elevation, which improves the manipulation of the drip chamber, as the user may securely grasp the drip chamber by the elevation with the hand. This grip ring is typically made of an opaque polymer material. It is molded onto the outer surface of the drip chamber using an molding process.

The grip ring is conventionally formed on the outer surface of the drip chamber in a separate step. This has disadvantages, in particular there is a very high effort to provide the three-part drip chambers conventionally manufactured by the complex injection molding process. The injection molding process, which is conventionally used to form the grip ring, is very complex in terms of equipment.

In the case of the drip chambers known in the prior art and available in the market, there entail considerable restrictions in the choice of materials used for the upper part and lower part, because these materials must be selected in such a way that they can be joined to each other safely and in the simplest possible way. In other words, the materials must be compatible with each other in terms of connectivity. In addition, the materials should also meet the requirements placed on a drip chamber, for example with regard to the transparency of the upper part and the elasticity of the lower part required for pumping.

Based on the above situations, there is a need of the invention to provide an improved two parts drip chamber for an infusion set. Further, it is also desired to provide a drip chamber which is inexpensive to manufacture, efficient, effective and simple in its construction and use.

### SUMMARY OF THE INVENTION

In one embodiment, two parts drip chamber of infusion set is disclosed. The drip chamber comprises a first part or an upper body with the proximal end is intended to be connected to a container or bottle of intravenous fluid and having piercing means extending radially outwards, wherein the piercing means is provided with hole for fluid to flow into the drip chamber from the fluid container or bottle to the patient. An air valve is situated at middle or lower middle portion of a first part or an upper body. The portion of distal end of a first part or an upper body is preformed in circular ring shape. The drip chamber further comprises a second part or a lower body with the distal end is intended to be connected to an intravenous infusion tubing and having fluid flow regulating element before connection point with intravenous tubing. The portion of the proximal end of second part or lower body is preformed in circular ring shape. The preformed circular ring shape region of first part or upper body and the preformed circular ring shape region of second part or lower body are intended to be securely couple in a leak-proof manner by adhesive material.

According to embodiment of the invention, a drip chamber of infusion set having a first part or an upper body as rigid or solid in nature and made up of the material selected from group of thermoplastic polymers containing Acrylonitrile butadiene styrene (ABS) or Polycarbonate (PC). A second part or a lower body is flexible in nature and made up of the material selected from group of plastic containing Thermoplastic elastomer (TPE), Thermoplastic Polyurethane (TPU) or Poly Vinyl Chloride (PVC). The preformed circular ring shape region of first part or upper body and the preformed circular ring shape region of second part or lower body are directly couple in a leak-proof manner by adhesive material selected from cyclohexanone or cyclohexanone with Poly Vinyl Chloride (PVC) resins. Once joined in leak-proof manner, the design of the preformed circular ring shapes of both first part or upper body and second part or lower body producing ergonomic elevated grip ring structure which improves the manipulation of the drip chamber, as the user may securely grasp the drip chamber by the elevation with the hand. Additionally, it is possible to give the grip ring a colour by adding colouring agent to adhesive material used to join preformed circular ring shapes of both first part or upper body and second part or lower body. By using a specific colouring agent, it is possible to individualize a drip chamber of a certain type or of a certain manufacturer in terms of identification. In this way, confusion in clinical use can be avoided.

According to embodiment of the invention, a drip chamber of infusion set where the distal end of a second part or a lower body comprises a fluid flow regulating element before connection point with intravenous infusion tubing. The fluid flow regulating element prevent the air from entering the flexible infusion line through the drip chamber.

The method according to the invention is a method for manufacturing two parts drip chamber. The method herein comprises the steps of: (A) providing a first part or upper body of the drip chamber with a region of distal end is preformed in circular ring shape; (B) providing a second part or lower body of the drip chamber with a region of proximal end is preformed in circular ring shape; and (C) coupling the first part or an upper body and second part or lower body with adhesive material selected from cyclohexanone or cyclohexanone with PVC resins, such that the preformed circular ring shape region of first part or upper body and the preformed circular ring shape region of a second part or a lower body are directly connected in a leak-proof manner and producing ergonomic elevated grip ring structure.

The two parts drip chamber of infusion set is engineered to function reliably and consistently, minimizing the risk of malfunctions that could compromise patient safety or healthcare worker effectiveness.

The two parts drip chamber of infusion set design allows for different sizes and configurations to accommodate various patient populations and clinical scenarios. This customization ensures that the two parts drip chamber of infusion set remains effective across diverse healthcare contexts.

Advantage of the two parts drip chamber according to the invention is that it provides supreme protection from air embolism and phlebitis. The two parts drip chamber is designed such that it not only prevents the entry of an air in the infusion line but also maintains a constant fluid level in case the container runs out of fluid.

Further areas of applicability of the present disclosure will become apparent from the detailed description provided hereinafter. It should be understood that the detailed description and specific examples, while indicating various embodiments, are intended for purposes of illustration only and are not intended to necessarily limit the scope of the disclosure.

### BRIEF DESCRIPTION OF THE DRAWINGS

The foregoing and other objects, features, and advantages of the invention will be apparent from the following detailed description taken in conjunction with the appended figures:

FIG. 1 illustrates a schematic view of two parts drip chamber of infusion set according to an embodiment of the present disclosure;

FIG. 2 illustrates an exploded view of two parts drip chamber according to an embodiment of the present disclosure;

FIG. 3 illustrates a flowchart of an embodiment of the method according to the invention.

In the appended figures, similar components and/or features may have the same reference label. Further, various components of the same type may be distinguished by following the reference label by a second alphabetical label that distinguishes among the similar components. If only the first reference label is used in the specification, the description is applicable to any one of the similar components having the same first reference label irrespective of the second reference label.

### DETAILED DESCRIPTION OF THE DRAWINGS

The ensuing description provides preferred exemplary embodiment(s) only, and is not intended to limit the scope, applicability or configuration of the disclosure. Rather, the ensuing description of the preferred exemplary embodiment(s) will provide those skilled in the art with an enabling description for implementing a preferred exemplary embodiment. It is understood that various changes may be made in the function and arrangement of elements without departing from the spirit and scope as set forth in the appended claims.

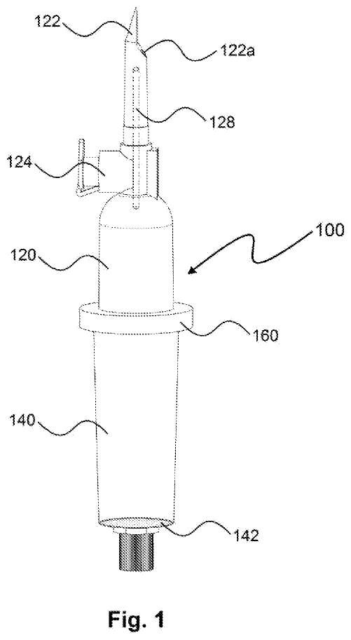

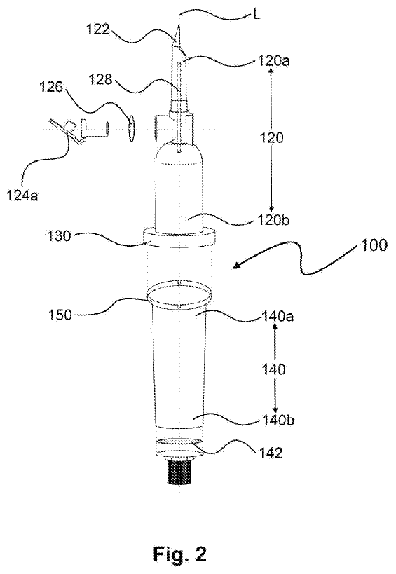

FIG. 1 and FIG. 2 show two parts drip chamber of infusion set according to present embodiment of the invention. The drip chamber (100) is shown in its operating position with the longitudinal axis L being vertical. In the following specification, the two parts of drip chamber “first part” (120) and “second part” (140) (“upper body” and “lower body”) are intended to simplify the description. The subject-matter of the invention is not thereby limited to a particular orientation of the drip chamber (100).

Referring to FIG. 1 and FIG. 2, which illustrating a schematic view and exploded view of two parts drip chamber (100) of infusion set. The drip chamber (100) comprises a first part or an upper body (120) with the proximal end (120a) is intended to be connected to a container or bottle of intravenous fluid. The proximal end (120a) having piercing means (122) in form of spike and is extending radially outwards. This spike is intended to be connected to a container for the liquid to be administered to the patient such that the liquid can pass from the container into the drip chamber (100), the container not being shown in the figures. The spike is provided with hole (122a) for fluid to flow into the drip chamber (100) from the container to the patient. The hole (122a) can be designed in any shape and size. The first part or an upper body (120) further comprises a drop former (128) and is provided in interior of the drip chamber (100). Fluid that enters the drip chamber (100) from the container through the spike enters the interior of the drip chamber (100) via the drop former (128). The drop former (128) is configured such that the fluid enters the drip chamber (100) in the form of drops of a standardized size. The first part or an upper body (120) further comprises an air valve (124) and is provided at middle or lower middle portion of a first part or an upper body (120). The air valve (124) comprises air filter (126) as well as a cap (124a). The function of the air valve (124) is, for example, that air is pumped into the container when the second part or a lower body (140) is compressed. This pumping function can initiate the flow of fluid from the container into the drip chamber. The air valve (124) allows air to enter the container for the fluid to be administered to the patient but does not allow a fluid to exit vice versa. The portion of distal end (120b) of a first part or an upper body (120) is preformed in circular ring shape (130).

The complete first part or an upper body (120) is transparent and rigid in nature. The rigid nature allows user to hold the first part or an upper body (120) and apply a force to pierce a container or bottle of intravenous fluid with spike. Entire first part or an upper body (120) is made up of material selected from group of thermoplastic polymers containing one or more of Acrylonitrile Butadiene Styrene (ABS) or Polycarbonate (PC).

In some of the embodiments, a first part or an upper body (120) is made up of material selected from group of thermoplastic polymers containing styrene based polymers and co-polymers such as styrene-acrylonitrile copolymer, methyl methacrylate acrylonitrile butadiene styrene, polyesters such as polyethylene terephthalate (PET), polypropylene, polyethylene and combination thereof.

In alternative embodiments not shown in the figures, other systems are provided to connect the first part or an upper body (120) of drip chamber (100) to the container, for example coupling systems which do not allow the drip chamber and the container to be separated once they have been connected.

The two part drip chamber (100) of infusion set further comprises a second part or a lower body (140) with the distal end (140b) is intended to be connected to an intravenous tubing to deliver intravenous fluid. The distal end (140b) is connecting in fluid tight manner with intravenous tubing, the intravenous tubing not being shown in the figures. The distal end (140b) is having fluid flow regulating element (142) provided just before the point at which distal end (140b) is intended to be connected to an intravenous tubing. Fluid that leaves the drip chamber (100) and enters into intravenous tubing by passing through the fluid flow regulating element (142). In case of the drip chamber (100) is empty, the fluid flow regulating element (142) ensure that intravenous tubing is still filled with fluid and hence prevents chances of air embolism and phlebitis. The portion of proximal end (140a) of a second part or a lower body (140) is preformed in circular ring shape (150).

The fluid flow regulating element (142) of the invention is a hydrophilic membrane, which is permeable to fluid and impermeable to air. The hydrophilic membrane has a circular shape. However, the hydrophilic membrane can have other geometrical shapes such as oval, semi-circular, square, rectangular, triangular, or combinations thereof. Preferably the material of hydrophilic membrane has property of flexibility and can be in form of sheet or film, tube or fiber, or plug form. The material can be a fabric, such as a nonwoven, woven, or knit fabric, or a scrim. The material can be made of paper such as filter paper, or a cloth, or a metal mesh. The material can also be a porous polymeric film or membrane, synthetic or natural, where the pores form the interstices or passageways. Representative polymers useful in the material include polyamide, nylon, polyurethane, polyester, polycarbonate, polyvinylidene fluoride, polyacrylic, polyolefins and the like.

Fluid flow regulating element (142), which as stated is a hydrophilic membrane, usually sealed over the infusion fluid outlet at distal end (140b) of second part or a lower body (140) of the drip chamber (100). The fluid flow regulating element (142) is arranged in the drip chamber (100) in a close fit manner eliminating the possibility of air being trapped or air bubbles being sucked into the infusion line. The fluid flow regulating element (142) can also be arranged by different means and methods over the infusion fluid outlet at distal end (140b) of second part or a lower body (140) of the drip chamber (100), for example, the fluid flow regulating element (142) can be arranged over a floating disc. The fluid flow regulating member is effective when wet.

In some embodiments, variable fluid flow rate is achieved using the two parts drip chamber (100) design of infusion set of present invention. Said variable fluid flow rate is achieved by fluid flow regulating element (142) located at a distal end (140b) of a second part or a lower body (140). The variable fluid flow rate is in range of 5 microns to 15 microns.

The complete second part or a lower body (140) is transparent and flexible in nature. The flexible nature allows user to perform pumping function to initiate the flow of fluid from the container into the drip chamber (100). Entire second part or a lower body (140) is made up of material selected from group of plastic containing one or more of Thermoplastic Elastomer (TPE), Thermoplastic Polyurethane (TPU) or Poly Vinyl Chloride (PVC).

In some of the embodiments, a second part or a lower body (140) is made up of material selected from group of thermoplastic elastomers such as thermoplastic copolyester elastomers, thermoplastic polyurethane elastomers, thermoplastic polyether block amide elastomers, SEBS (Styrene Ethylene Butylene Styrene), styrene-isoprene-styrene block copolymer, styrene- butadiene-styrene block copolymer, styrene-butadiene copolymer material, soft polybutadiene, soft polyvinyl chloride or soft styrene elastomer, polyethylene, ethyl vinyl acetate (EVA), polydimethylsiloxane (PDMS), cyclic olefin copolymer (COC), cyclic olefin polymer (COP) and combination thereof.

The two part drip chamber (100) of infusion set of present invention, wherein preformed circular ring shape region (130) of a first part or an upper body (120) and the preformed circular ring shape region (150) of a second part or a lower body (140) are directly connected in a leak-proof manner by adhesive material selected from one or more of Cyclohexanone or Cyclohexanone with Poly Vinyl Chloride (PVC) resins. Once joined in leak-proof manner, the design of the preformed circular ring shapes of both upper body (120) and lower body (140) producing ergonomic elevated grip ring structure (160) which improves the manipulation of the drip chamber (100), as the user may securely grasp the drip chamber (100) by the elevation with the hand. Additionally, it is possible to give the grip ring a colour by adding colouring agent to adhesive material used to join preformed circular ring shapes of both upper body (120) and lower body (140). By using a specific colouring, it is possible to individualize a drip chamber (100) of a certain type or of a certain manufacturer in terms of identification. In this way, confusion in clinical use can be avoided.

In some of the embodiments, adhesive material selected from ethyl acetate, methylene chloride, tetrahydrofuran, methyl ethyl ketone, medical grade epoxy adhesives, polyurethane adhesives, silicone adhesives, UV curable adhesives and combination thereof.

The diameter of preformed circular ring shape region (130) of a first part or an upper body (120) is greater than diameter of preformed circular ring shape region (150) of a second part or a lower body (140). However, in some embodiments, the diameter of preformed circular ring shape region (130) of a first part or an upper body (120) is lesser than diameter of preformed circular ring shape region (150) of a second part or a lower body (140).

In some embodiments the transparency of either a first part or an upper body (120) or second part or a lower body (140) or both can be varied from 10% to 90% to allow visible light to pass through and for ease of proper operation of infusion set.

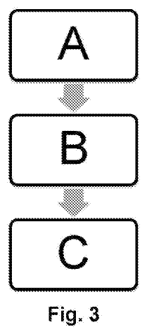

Referring to FIG. 3, it illustrates flow chart of an embodiment of the method according to the invention for manufacturing two parts drip chamber. In step (A), a first part or an upper body is provided. A first part or an upper body is intended to be connected to a container or bottle of intravenous fluid. Further the distal end of a first part or an upper member is intended to be connected to proximal end of a second part or a lower member. Additionally, region of the distal end of a first part or an upper member is preformed in circular ring shape. Further the first part or an upper body (120) which is transparent and solid in nature and made up of material selected from group of thermoplastic polymers containing one or more of Acrylonitrile Butadiene Styrene (ABS) or Polycarbonate (PC).

In the subsequent step (B), a second part or a lower body is provided. A second part or a lower body is intended to be connected to intravenous infusion tube. Further the proximal end of a second part or a lower body is intended to be connected to distal end of a first part or an upper body. Additionally, region of the proximal end of a second part or lower body is preformed in circular ring shape. Further the second part or a lower body (140) which is transparent and flexible in nature and made up of material selected from group of plastic containing one or more of Thermoplastic Elastomer (TPE), Thermoplastic Polyurethane (TPU) or Poly Vinyl Chloride (PVC).

In the subsequent step (C), a first part or an upper body and a second part or a lower body are directly coupled to each other in leak-proof manner. According to an embodiment of the method according to the invention, in step (C), a first part or an upper body and a second part or a lower body are securely coupled to each other by using adhesive material selected from one or more of Cyclohexanone or Cyclohexanone with Poly Vinyl Chloride (PVC) resins and coupling of both parts is carried out in such a way that the preformed circular ring shape region of a first part or an upper body and the preformed circular ring shape region of a second part or a lower body are directly coupled in a leak- proof manner for producing ergonomic elevated grip ring structure which improves the manipulation of the drip chamber with the user hand. The distal end of a first part or an upper body is intended to be connected to proximal end of a second part or a lower body. Additionally, it is possible to give the grip ring structure a colour by adding colouring agent to adhesive used to join preformed circular ring shapes of both upper body and lower body. By using a specific colouring, it is possible to individualize a drip chamber of a certain type or of a certain manufacturer in terms of identification. In this way, confusion in clinical use can be avoided.

In alternative embodiments not shown in the figures, the same steps are performed in other sequences. Herein, different sequences are possible as long as it is ensured that the parts to be joined to each other are provided in time to be joined to each other.

As an alternative to the described coupling method, other coupling methods are in principle also possible in other embodiments, depending on the nature of the materials, for example gluing using an adhesive, for example preferably UV gluing, in particular by means of acrylate adhesive, or welding such as mirror welding, torsion welding, and preferably, ultrasonic welding.

While particular embodiments have been described and illustrated, it should be understood that the scope of the disclosure is not limited to these specific examples. Variations and modifications within the scope of the appended claims will be readily apparent to those skilled in the art. Therefore, the present disclosure is not to be limited to the embodiments disclosed herein but is to be accorded the widest scope consistent with the principles and novel features disclosed herein.

Accordingly, the present disclosure should not be construed as limited by the foregoing description, but rather construed in accordance with the appended claims.

Specific details are given in the above description to provide a thorough understanding of the embodiments. However, it is understood that the embodiments may be practiced without these specific details.

Also, it is noted that the embodiments may be described as a process which is depicted as a schematic representation. Although a depiction may describe the operations as a sequential process, many of the operations can be performed in parallel or concurrently.

The systems and devices discussed herein are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims. Additionally, the techniques discussed herein may provide differing results with different types of context awareness classifiers.

Unless defined otherwise, all technical and scientific terms used herein have the same meaning as commonly or conventionally understood. As used herein, the articles “a” and “an” refer to one or to more than one (i.e., to at least one) of the grammatical object of the article. By way of example, “an element” means one element or more than one element. “About” and/or “approximately” as used herein when referring to a measurable value such as an amount, a temporal duration, and the like, encompasses variations of ±20% or ±10%, ±5%, or ±0.1% from the specified value, as such variations are appropriate to in the context of the systems, devices, circuits, methods, and other implementations described herein. “Substantially” as used herein when referring to a measurable value such as an amount, a temporal duration, a physical attribute (such as frequency), and the like, also encompasses variations of ±20% or ±10%, +5%, or ±0.1% from the specified value, as such variations are appropriate to in the context of the systems, devices, circuits, methods, and other implementations described herein.

As used herein, including in the claims, “and” as used in a list of items prefaced by “at least one of” or “one or more of” indicates that any combination of the listed items may be used. For example, a list of “at least one of A, B, and C” includes any of the combinations A or B or C or AB or AC or BC and/or ABC (i.e., A and B and C). Furthermore, to the extent more than one occurrence or use of the items A, B, or C is possible, multiple uses of A, B, and/or C may form part of the contemplated combinations. For example, a list of “at least one of A, B, and C” may also include AA, AAB, AAA, BB, etc.

### LIST OF REFERENCE NUMERALS

100 Two parts drip chamber
120 First part or an upper body
120a Proximal end of a first part or an upper body
120b Distal end of a first part or an upper body
122 Piercing means
122a Hole
124 Air Valve
124a Cap
126 Air Filter
128 Drop former
130 Preformed circular ring shape of a first part or an upper body
140 Second part or a lower body
140a Proximal end of a second part or a lower body
140b Distal end of a second part or a lower body
142 Fluid flow regulating element
150 Preformed circular ring shape of a second part or a lower body
160 Ergonomic elevated grip structure

1. A drip chamber for an infusion set to administer delivery of intravenous fluid to a patient, wherein the drip chamber comprises:
an upper body which is transparent and made up of a material selected from a group of thermoplastic polymers containing one or more of Acrylonitrile Butadiene Styrene (ABS) or Polycarbonate (PC), wherein:
a proximal end of the upper body includes a piercing means extending radially outwards for deflating a container or bottle of intravenous fluid, wherein the piercing means is provided with hole for fluid to flow into the drip chamber;
an air valve with a cap is situated at a middle or lower middle portion of the upper body, wherein the air valve has a filter and used for management of air in the drip chamber;
a drop former is situated in the upper body of an interior of the drip chamber; and
a distal end region of the upper body is preformed in a circular ring shape; and
a lower body which is transparent and made up of a material selected from a group of plastic containing one or more of Thermoplastic Elastomer (TPE), Thermoplastic Polyurethane (TPU) or Poly Vinyl Chloride (PVC), wherein:
a distal end of the lower body is configured to connect to an intravenous infusion tube, wherein the distal end (140b) of a the lower body includes a fluid flow regulating element before a connection point with the intravenous infusion tube; and
a proximal end region of the lower body is preformed in a circular ring shape;
wherein the preformed circular ring shape region at the distal end of the upper body (120) and the preformed circular ring shape region at the proximal end of the lower body are securely coupled in a leak-proof manner by an adhesive material selected from one or more of Cyclohexanone or Cyclohexanone with Poly Vinyl Chloride (PVC) resins.

2. The drip chamber as claimed in claim 1, wherein a leak-proof secure connection between the preformed circular ring shape regions of both the upper body and the lower body produces an ergonomic elevated grip structure which improves manipulation of the drip chamber with a user's hand.

3. The drip chamber as claimed in claim 1, wherein a diameter of the preformed circular ring shape region of the upper body is greater than a diameter of the preformed circular ring shape region of the lower body.

4. The drip chamber as claimed in claim 1, wherein a diameter of the preformed circular ring shape region of the upper body is lesser than a diameter of the preformed circular ring shape region of the lower body.

5. The drip chamber as claimed in claim 1, wherein the upper body and/or the lower body are flexible.

6. The drip chamber as claimed in claim 1, wherein the upper body is rigid and the lower body is flexible.

7. The drip chamber as claimed in claim 1, wherein the upper body is flexible and the lower body is rigid.

8. The drip chamber as claimed in claim 1, wherein the fluid flow regulating element is a hydrophilic membrane that is permeable to fluid and impermeable to air.

9. A method of manufacturing a drip chamber of an infusion set comprising the steps of:
a) providing an upper body of the drip chamber, wherein a distal end region of the upper body is preformed in a circular ring shape;
b) providing a lower body of the drip chamber, wherein a proximal end region of the lower body is preformed in a circular ring shape; and
c) securely coupling the upper body and the lower body with an adhesive material selected from one or more of Cyclohexanone or Cyclohexanone with Poly Vinyl Chloride (PVC) resins,
wherein the preformed circular ring shape regions of both the upper body and the lower body are securely coupled in a leak-proof manner, thereby producing an ergonomic elevated grip structure which improves manipulation of the drip chamber with a user's hand.
